(19) United States
(12) Patent Application Publication
(10) Pub. No.: US 2022/0283177 A1
(43) Pub. Date: Sep. 8, 2022

(54) METHOD FOR DETECTING INTERACTION AND AFFINITY BETWEEN LIGAND AND PROTEIN

(71) Applicant: DALIAN INSTITUTE OF CHEMICAL PHYSICS, CHINESE ACADEMY OF SCIENCES, Dalian (CN)

(72) Inventors: Mingliang YE, Dalian (CN); Xiaolei ZHANG, Dalian (CN); Lianghai HU, Dalian (CN)

(21) Appl. No.: 17/664,401

(22) Filed: May 20, 2022

Related U.S. Application Data
(63) Continuation-in-part of application No. PCT/CN2020/130079, filed on Nov. 19, 2020.

(30) Foreign Application Priority Data
Nov. 20, 2019 (CN) 201911140968.3

(51) Int. Cl. G01N 33/68; G01N 33/58

(52) U.S. Cl. CPC G01N 33/6845; G01N 33/6842; G01N 33/58

(57) ABSTRACT

A method of solvent-induced protein precipitation (SIP) for detecting the interaction of ligands with proteins in a complex protein sample. After the equal amount of solvent is added to the protein samples with and without a ligand to denature and precipitate the proteins, the protein abundances in supernatant and/or precipitate in the ligand group and the control group are measured by quantitative technology. The target protein(s) of a ligand is/are determined by comparing the differences of protein abundances in the ligand group and the control group. The affinity between a ligand and its targets can be evaluated by dose dependent experiments. This method does not require the chemical modification of the ligand and has the feature of high specificity. Furthermore, in certain embodiments, the targets identified by SIP method are complementary to those identified by thermal proteome profiling (TPP) method.

### FIELD OF THE INVENTION

The present invention relates to the field of drug target discovery based on the proteomics research. Especially, the present invention is related to the establishment of a method for target identification and affinity evaluation based on solvent-induced protein precipitation.

### BACKGROUND OF THE INVENTION

The identification of drug target proteins is an important part in drug development. The discovery of novel targets can provide breakthroughs in drug identification and offer an important theoretical basis for the discovery and design of lead compounds (Bantscheff M, et al, Nature Biotechnology, 2007, 25: 1035-1044). Identification of the potential drug targets by means of proteomics or biotechnology is of great significance for studying the molecular mechanisms, side effects and other medical value of drugs. At present, a series of proteomics-based methods for identifying drug target proteins have been developed, which are mainly divided into two categories: small molecule modification/immobilization mode and modification-free drug mode. The former includes activity-based probe analysis (ABPP) (Van Esbroeck ACM, et al, Science, 2017, 356: 1084-1087) and affinity chromatography (Hoehenwarter W, et al, Molecular & Cellular Proteomics, 2013, 12: 369-380). The requirement of chemical modification for a compound in this strategy is a bottleneck in targets identification of drugs. The limitations mainly include: 1) Modification or immobilization of the compounds will usually alter the physicochemical properties and permeability into biofilms, resulting in high false positive identification of targets. 2) This method is not suitable for the target identification of the weak interaction with a drug. The ideal method for identifying target proteins is without any chemical modification of a compound, and does not dependent on the affinity. Therefore, energetics difference-based strategy for target identification of ligands has received more and more attention in recent years.

The conformation of a protein will change after binding with a ligand, indicating that the protein is in a different energy state. Energetics difference-based modification-free strategy for target identification was based on the concept that the protein become more stable upon drug binding, which is more tolerant to heat, oxidative denaturation and proteolysis. The target protein(s) of a ligand is/are determined by comparing the abundance differences of protein with and without binding a ligand. This method does not require any modification/immobilization of the compounds, unlike traditional affinity-based capture method. The energetics difference-based strategy for target identification of ligands mainly includes stability of proteins from rates of oxidation (SPROX) (Strickland E C, et al, Nature Protocols, 2013, 8: 148-161), drug affinity responsive target stability assay (DARTS) (Chin R M, et al, Nature, 2014, 510: 397-401; Lomenick B, et al, PNAS, 2009, 106: 21984-21989), limited proteolysis (LIP) (Feng Y H, et al, Nature Biotechnology, 2014, 32: 1036-1044), cellular thermal shift assay (CETSA) and thermal proteome profiling (TPP) (Savitski M M, et al, Science, 2014, 346: 1255784; Huber K V M, et al, Nature Methods, 2015, 12: 1055-1057). The above methods provide new insights for the identification of targets of ligands, however, there still have some drawbacks for each method. The SPROX method is based on the difference in the oxidation rate of methionine to identification targets of ligands. Due to the fact that the target identification is through assessing the hydrogen peroxide-mediated oxidation rates of methionine residues, the protein coverage is limited. The LIP method is an improvement of the DRATS method, which have disadvantages such as the increasing complexity of the sample, high requirements for peptide quantification, accuracy and precision. The TPP method does not require identification of specific peptides, and the protein coverage is similar to the traditional bottom-up method. However, some proteins with small thermal stability shifts and poorly fitted “S” curve will be discarded when analyzing the data of thermal stability change, resulting in the loss of potential targets of a ligand.

In conclusion, although some methods for drug target identification have been developed in recent years, their applicability and sensitivity still need to be improved. Existing methods do not exploit the principle of solvent-induced proteins precipitation for the target identification of a ligand. The present invention proposes a novel method for identification and/or screening of drug targets based on solvent-induced proteins precipitation.

### SUMMARY OF THE INVENTION

The present invention relates to energetics-based methods that rely on the fact that the ligand binding proteins have a higher resistance to the solvent-induced precipitation (SIP) for target proteins identification of a ligand, and to the method for determining the affinity of targets interacting with a ligand. Solvent precipitates proteins by decreasing dielectric constant and competing for protein hydration, which is different from thermal denaturation-induced proteins precipitation (such CETSA and TPP methods).

In one aspect, the present invention of SIP method does not require the immobilization or modification of the ligands, thereby overcoming the shortcomings such as the affinity or specific change of ligands and high false positive rate for target identification. The method can be applied for any proteome investigation for target identification. In another aspect, the method can not only identify the target(s) and off-target(s) interacted with a ligand, but also can evaluate affinity of drug-target interaction. Furthermore, in certain embodiments, target spaces identified by different precipitation approaches are complementary.

### DETAILED DESCRIPTION OF THE INVENTION

The present invention is related to the method based on solvent-induced precipitation (SIP) for target proteins identification and affinity determination of a ligand. This method has the characteristics of high throughput, low cost and high specificity. Moreover, it can overcome the shortcomings of traditional affinity-based method with high false positives and unsuitable for identification of weak interactions. The SIP method exploits the tolerance difference of protein with and without a ligand to solvent-induced protein precipitation for target identification and affinity evaluation. The target proteins binding a test ligand will become more resistant to the denaturation and precipitation induced by solvent treatment. After the equal amount of solvent is added to the protein samples with and without a ligand to denature and precipitate the proteins, the protein abundances of supernatant and/or precipitate in the ligand group and the control group are measured by quantitative technology. The target protein(s) of a ligand is/are determined by comparing the differences of protein abundances in the ligand group and the control group.

The method is provided for identifying the target proteins of ligands in various embodiments. The method comprises, in one alternative: (a) The protein solution incubated with a ligand is used as ligand group, the protein solution incubated with equivalent amount of ligand dissolving solvent in the absence of ligand is used as the control group; (b) Add an equal amount of denaturing solvent to the ligand group and the control group to initiate protein denaturation and resulting in precipitation. (c) Quantify the abundance of each protein in supernatant and/or precipitate of the ligand group and the control group. (d) Compare the abundance difference of each protein in the ligand group and the control group (that is, the difference in the abundance of the same protein in the supernatant and/or precipitate) to determine the target(s) of a ligand. The soluble protein (supernatant) is separated from the precipitate by centrifugation before the quantification in step (c).

The protein solution includes one protein or a mixture of two or more proteins; the protein mixture includes one or more of cell or tissue extracts; the cell or tissue extract is derived from one or more of humans, animals, plants or bacteria. The protein solution includes one or more of blood or plasma; the blood or plasma is obtained from one or more of humans or animals.

Mild extraction condition is performed for protein solution preparation, allowing proteins in one or more cells or tissues to maintain the natural conformation (the specific spatial structure of the protein in living cells or tissues). Preferably, the extraction condition includes, but not limit to, PBS (phosphate buffer saline) alone or PBS supplemented with 0.2-0.4% NP-40 (Nonidet P 40) as a buffer, combining with 2-5 freeze-thaw cycles in liquid nitrogen. The thawing temperature is at 10-50° C.

The ligand includes one or more of drugs, metabolites, natural productions or plant extracts, food additives, environmental pollutants, agriculture pesticides or herbicides, environmental agents, metal ions, nanoparticles, peptides, proteins and other substances that may interact with proteins.

The protein solution is divided into two groups in step (a), one group is added with a ligand as the ligand group, and the other group is without ligand as the control group. Or, but not limited to two groups. The ligand group can perform more than two groups of protein samples with different concentrations of ligand, and the control group can apply blank (that is, without ligand(s)) or other ligand(s) with similar structure(s) and different target proteins.

The solvent for protein denaturation is one or a mixture of two or more solvents. The solvent include one or more of organic or inorganic substances that can denature and precipitate proteins. The solvent includes but not limited to one or more of solvents, acidic agents, alkaline agents, metal ions or salts. The solvent includes, but not limited to, one or two of acetone, methanol, ethanol, acetic acid, ascorbic acid (Vc), citric acid (CA), and trifluoroacetic acid. Preferably, the solvent mixture includes, but is not limited to, a mixture of three solvents (acetone, ethanol and acetic acid) (abbreviated as A. E. A. or A. A. A.). The volume ratio of solvent mixture is acetone:ethanol:acetic acid=50:50:0.1.

The solvent window used for protein denaturation and precipitation in the ligand group and the control group can be appropriately adjusted according to the different solvents. The principle of solvent window selection is that the solvent or solvent mixture can cause the initial precipitation of the protein to the range of 80-90% of the protein precipitation. Preferably, the final volume percentage of the solvent mixture A.E.A. (or A.A.A.) within the range of 9%-22%; the final concentration of Vc is within the range of 1-15 mM; or, the final concentration of CA is within the range of 1-5 mM. Combinations of solvent mixtures include kit development. The equilibrium condition of the solvent-treated protein solution can be adjusted appropriately (that is, shaking at 20-30° C. for 20-40 min or shaking at 30-40° C. for 10-20 min to achieve the purpose of partial protein denaturation in protein solution). Preferably, the condition of reaction equilibrium is shaking at 800 rpm for 20 min at 37° C.

Protein abundance can be detected in the soluble fraction (i.e., the supernatant) or precipitate, or in both. To quantify the protein abundance in the ligand group and the control group after the solvent(s) treatment in step (c) include, but are not limited to western blotting and quantitative proteomics technology. The labeling methods of peptides in quantitative proteomics technology include label-free quantification and/or label quantification. The methods of labeling quantification include one or more of dimethyl labeling and multiplex isotope labeling methods such as TMT (neutron-encoded isobaric tandem mass tags) or ITRAQ (isobaric tags for relative and absolute quantification). In addition to stable isotope labeling based methods, proteins could also be quantified by label free methods including data independent acquisition (DIA) for target discovery and affinity determination.

There are several analytical assays to obtain the stabilization shift of protein. For the dimethyl-labeled samples, the criterion for target protein identification is that the difference in the abundance or relative abundance distance of each protein in the ligand group and the control group is ≥ or ≤ to a certain threshold. The threshold can be adjusted appropriately according to the different ligands. The optimal threshold is determined by maximizing the sensitivity and specificity, such as the fold change of protein abundance ≥2. The protein is stabilized (that is, the protein abundance of supernatant in ligand group is higher than that in the control group or the protein abundance of supernatant in ligand group is lower than that in the control group) by binding with a ligand and consider it as the directly target. In contrast, the protein is destabilized (that is, the protein abundance of supernatant in ligand group is lower than that in the control group or the protein abundance of precipitation in ligand group is higher than that in the control group) by binding with the ligand and consider it as the indirectly target. For the TMT or ITRAQ multiple labeling experiment, the screening criteria of target proteins is by comparing the difference in distances sum (ΔDistance) of the same protein in the ligand group and the control group at five denaturation points. Regardless of the above-mentioned labeling or data processing methods, the threshold of the abundance ratio or ΔDistance is not fixed. In theory, the stricter the threshold, the higher the confidence of the target protein identification.

The present invention also provides a method to determine the affinity of drug-target interaction in complex protein solutions by using dose responsive experiment. The method comprises, in one alternative: (a) Incubate the same ligand with different concentration gradients with the tested protein solution separately (usually choose 5 or more than 6 final concentrations for the ligand, one of which is the control point without ligand, that is, the final concentration of the ligand at the control point is 0); (b) Add an equal amount of solvent (preferably the same final concentration) to the mixture containing proteins and ligand to precipitate the proteins; (c) Quantify the abundance of each protein in supernatant and/or precipitate containing proteins and ligand. (d) Calculate the concentration for 50% of maximal effect (i.e. EC50) to obtain affinity between the ligand and the target protein, by taking the abscissa as the different drug concentrations and the ordinate as the protein abundance to fit curve. The calculation equation: Y=min+(max−min)/(1+10\hat{ }((Log EC50−X)*Hill Slope)), Y is the protein abundance, X is the different drug concentrations, min and max are the minimum and maximum values of the corresponding protein abundance on the Y axis, respectively. Hill Slope is the absolute value of the maximum slope of the curve (i.e., the midpoint of the curve).

The soluble protein (supernatant) is separated from the precipitate by centrifugation before the quantification in step (c).

The protein solution includes one protein or a mixture of two or more proteins; the protein mixture includes one or more of cell or tissue extracts; the cell or tissue extract is derived from one or more of humans, animals, plants or bacteria. The protein solution includes one or more of blood or plasma; the blood or plasma is derived from one or more of humans or animals.

Mild extraction condition is performed for protein solution preparation, allowing proteins in one or more cells or tissues to maintain the natural conformation (the specific spatial structure of the protein in living cells or tissues). Preferably, the extraction condition includes, but not limit to, PBS (phosphate buffer saline) alone or PBS supplemented with 0.2-0.4% NP-40 (Nonidet P 40) as a buffer, combining with three freeze-thaw cycles in liquid nitrogen. The thawing temperature is at 10-50° C.

The ligand includes one or more of drugs, metabolites, natural productions or plant extracts, food additives, environmental pollutants, agriculture pesticides or herbicides, environmental agents, metal ions, nanoparticles, peptides, proteins and other substances that may interact with proteins.

The protein solution is divided into two groups in step (a), one group is added with a ligand as the ligand group, and the other group is without ligand as the control group. Or, but not limited to two groups. The ligand group can perform more than two groups of protein samples with different concentrations of ligand, and the control group can apply blank (that is, without ligand(s)) or other ligand(s) with similar structure(s) and different target proteins.

The solvent for protein denaturation is one or a mixture of two or more solvents. The solvent include one or more of organic or inorganic substances that can denature and precipitate proteins. The solvent includes but not limited to one or more of solvents, acidic agents, alkaline agents, metal ions or salts. The solvent includes, but not limited to, one or two of acetone, methanol, ethanol, acetic acid, ascorbic acid (Vc), citric acid (CA), and trifluoroacetic acid. Preferably, the solvent mixture includes, but is not limited to, a mixture of three solvents (acetone, ethanol and acetic acid) (abbreviated as A. E. A. or A. A. A.). The volume ratio of solvent mixture is acetone:ethanol:acetic acid=50:50:0.1.

The solvent window used for protein denaturation and precipitation in the ligand group and the control group can be appropriately adjusted according to the different solvents. The principle of solvent window selection is that the solvent or solvent mixture can cause the initial precipitation of the protein to the range of 80-90% of the protein precipitation. Preferably, the final volume percentage of the solvent mixture A.E.A. (or A.A.A.) within the range of 9%-22%; the final concentration of Vc is within the range of 1-15 mM; or, the final concentration of CA is within the range of 1-5 mM. Combinations of solvent mixtures include kit development. The equilibrium condition of the solvent-treated protein solution can be adjusted appropriately (that is, shaking at 20-30° C. for 20-40 min or shaking at 30-40° C. for 10-20 min to achieve the purpose of partial protein denaturation in protein solution). Preferably, the condition of reaction equilibrium is shaking at 800 rpm for 20 min at 37° C.

Protein abundance can be detected in the soluble fraction (i.e., the supernatant) or precipitate, or in both. To quantify the protein abundance in the ligand group and the control group after the solvent(s) treatment in step (c) include, but are not limited to western blotting and quantitative proteomics technology. The labeling methods of peptides in quantitative proteomics technology include label-free quantification and/or label quantification. The methods of labeling quantification include one or more of dimethyl labeling and multiplex isotope labeling methods such as TMT (neutron-encoded isobaric tandem mass tags) or ITRAQ (isobaric tags for relative and absolute quantification). In addition to stable isotope labeling based methods, proteins could also be quantified by label free methods including data independent acquisition (DIA) for target discovery and affinity determination.

### Advances of the Invention

### Advantages of the Present Invention Include:

(1) The high specific and throughput of the SIP method in target identification. And the ligand used for target identification by SIP method does not require modification, which overcomes the difficulties existing in traditional chemical proteome-based methods for target identification, including the changes in physicochemical properties and biomembrane permeability of the ligand, the high false positive rate, as well as the unsuitability for targets identification for weak interactions with drugs. In certain embodiments, the known target protein DHFR of MTX is not only screened o have a significant stability change by the SIP method, but the stability change of this protein has the highest ranking. In addition, the characteristic of high throughput of SIP method makes it possible to identify not only the desired target(s) for a ligand, but also the off-target(s) interacted with test ligand in the proteome scale. In Example 5, in addition to the identification of known target heat shock 90 family proteins of geldanamycin, a subunit of complex I in the mitochondrial respiratory chain, NDUFV1, was identified as an off-target protein of geldanamycin.

(2) The ability for SIP method to determine the affinity of ligands to known and unknown target proteins. In Examples 6, 14 and 15), the affinities between geldanamycin and the known target protein HSP90AB1, as well as MTX and DHFR, were accurately assessed using the SIP method, all of which were similar to the known affinities. In addition, the SIP method was used to evaluate the affinity between geldanamycin and the novel off-target protein NDUFV1 screened by SIP method of the present invention. The ability of SIP method for evaluating the affinity of ligand-target interaction offers the sufficient information for the investigation of mechanism of a ligand and drug discovery.

(3) The choice of solvents is wide. Solvents include organic or inorganic substances that can denature and precipitate proteins, including but not limited to organic solvents, acidic reagents, basic reagents, metal ions or salts. And the solvent may be one or a combination of two or more of the above solvents, but is not limited to these solvents.

(4) Target proteins identified by SIP method and TPP method are complementary. Savitski et al (Science, 2014, 346, 1255784) have performed a comprehensive study to screen the targets of pan-kinase inhibitor staurosporine by TPP approach. In Examples 4 and 13, the quantified kinases and the identified protein kinase targets binding with staurosporine between SIP and TPP methods were compared. It was found that several protein kinases had significant stabilization shifts in both TPP and SIP. There were some protein kinases showed significant stabilization only in SIP approach but not in TPP method. Thus, above observation indicated that target proteins identified by different precipitation methods are complementary.

(5) The present method of this invention can be applied to screen the target proteins of diverse ligands. The ligands include drugs, metabolites, natural productions or plant extracts, food additives, environmental pollutants, agriculture pesticides or herbicides, environmental agents, metal ions, nanoparticles, peptides, proteins and other substances that may interact with proteins. This method provide an unbiased and complementary tool for screening of target(s) of diverse ligands.

### BRIEF DESCRIPTION OF THE FIGURES

The present invention will be better understood with the detail description given herein and from the accompanying drawings, which are given by the way of illustration only and do not limit the intended scope of the invention.

FIG. 1 show the schematic representation of solvent-induced protein precipitation (SIP) approach for the screening of drug target proteins.

FIG. 2A to FIG. 2F show the identification of known targets of MTX and SNS-032 by solvent mixture-based SIP approach. FIG. 2A: Western blotting confirming the stabilization of DHFR by MTX in 293T cell lysate. FIG. 2B: Scatter plot of fold change of DHFR protein abundance in 13% A.E.A.-treated samples. FIG. 2C: Scatter plot of fold change of DHFR protein abundance 15% A.E.A.-treated samples. The target hits were obtained by LC-MS/MS data from two replicate runs. FIG. 2D: Western blotting confirming the stabilization of CDK9 by SNS-032 in 293T cell lysate. FIG. 2E: Scatter plot of fold change of CDK9 protein abundance in 12% A.E.A.-treated samples. FIG. 2F: Scatter plot of fold change of the GSK-3a in 13% A.E.A.-treated samples.

FIG. 3A to FIG. 3C show the identification of protein kinase targets of broad-specificity inhibitor staurosporine by solvent mixture-based SIP approach. FIG. 3A: Scatter plot of fold change of kinase abundances in 15% A.E.A.-treated samples staurosporine experiment. FIG. 3B: Scatter plot of fold change of kinase abundances in 16% A.E.A.-treated samples staurosporine experiment. FIG. 3C: Scatter plot of fold change of kinase abundances in 17% A.E.A.-treated samples of staurosporine experiment.

FIG. 4A to FIG. 4C show the comparison between solvent mixture-based SIP in present invention and TPP in the reference (M. M. Savitski, et al., (2014) Science 346: 1255784) on protein kinases binding with staurosporine. FIG. 4A: Total proteins quantified by in the two studies with different proteomics analysis workflows. FIG. 4B: Venn diagrams showing overlap of total quantified protein kinases (left) and the stabilized protein kinases (right) between solvent mixture-based SIP and Savitski-TPP. FIG. 4C: The comparison of protein kinases with stabilization shifts either in SIP or TPP for the 15 kinases quantified both in solvent mixture-based SIP and Savitski-TPP.

FIG. 5A to FIG. 5E show the discovery of protein targets of geldanamycin by solvent mixture-based SIP method and validation of screened potential off-target protein NDUVF1 through western blotting. FIG. 5A: Western blotting confirmed the stabilization of geldanamycin by HSP90AB1 in HeLa cell lysate. FIGS. 5B, 5C, and 5D are scatter plot of fold change of HSP90AB1 protein abundance in 15%, 16%, and 17% A.E.A.-treated samples, respectively. FIG. 5E: Western blotting confirming the stabilization of geldanamycin by NDUFV1 protein in HeLa cell lysate.

FIG. 6A and FIG. 6B show the affinity evaluation for ligand-protein interaction through using solvent mixture-based SIP approach. FIG. 6A: Affinity of geldanamycin to target protein HSP90AB1; and FIG. 6B: candidate protein NDUFV1 was estimated in HeLa lysate after incubating with different concentrations of geldanamycin at 15% A.E.A. concentration by using western blotting.

FIG. 7A and FIG. 7E show the GO analysis of candidate protein targets of geldanamycin identified by solvent mixture-based SIP approach and potential mechanism of geldanamycin-induced hepatotoxicity. FIG. 7A presents analysis of candidate protein targets that binding with geldanamycin on biological process; FIG. 7B shows molecular function; and FIG. 7C presents cellular component using online tool STRING. FIG. 7D: The pathways analysis of candidate protein targets by using online tool Reactome. FIG. 7E: The mechanism of geldanamycin-induced hepatotoxicity may be mainly due to multiple factors including mitochondrial respiratory chain disorder, ROS production accumulation, metabolism disorder and damage in liver development. Fork symbol represented the identified off-targets in present study through using solvent mixture-based SIP approach.

FIG. 8A to FIG. 8H show acidic agent-based SIP approach to identify the targets of small molecules MTX and SNS-032. FIG. 8A: Western blotting confirmed MTX stabilized the known target DHFR in 293T cell lysate after treating with concentration gradients of Vc (ascorbic acid). The known target DHFR of MTX was identified at 6 mM in FIG. 8B and 8 mM in FIG. 8C Vc-treated samples based on the proteomic readout. FIG. 8D Western blotting confirmed MTX stabilized the known target DHFR in 293T cell lysate after treating with different concentration gradients of CA (ascorbic acid). The known target DHFR of MTX was identified at 3 mM in FIG. 8E and 3.5 mM in FIG. 8F CA-treated samples by quantitative proteomics. FIG. 8G: Western blotting confirmed SNS-032 stabilized the known target CDK9 in 293T cell lysate after treating with concentration gradients of CA. FIG. 8H The another known target CDK2 of SNS-032 was identified at 3 mM CA-treated samples by quantitative proteomics. The target hits were obtained by LC-MS/MS data from two replicate runs.

FIG. 9A and FIG. 9B show the comparison of the staurosporine-induced stabilized target proteins identified by CA-based SIP and Savitski-TPP approaches in total cell lysate. FIG. 9A: identification of protein kinase in 293 Tcell lysate by CA-based SIP approach. The red dots represent the quantified protein kinases. FIG. 9B: Comparison of the number of staurosporine-induced stabilized proteins identified by CA-based SIP and Savitski-TPP approaches. A total of 13 stabilized protein kinases were identified only by CA-based SIP and not by TPP approach.

FIG. 10A and FIG. 10B show the examples of stability shift curves of stabilized proteins kinases binding with staurosporine identified by CA-based SIP approach. FIG. 10A: Examples of stability shift curves for staurosporine-induced stabilized proteins commonly identified in CA-SIP and Savitski-TPP experiments. FIG. 10B: Examples of stability shift curves for staurosporine-induced stabilized proteins only identified in CA-SIP but not in Savitski-TPP experiments.

FIG. 11A and FIG. 11B show the affinity evaluation for ligand-protein interaction by using acidic agent-based SIP approach. Drug dose-dependent profile of the target protein DHFR on MTX in 293T cell lysate treated with 12 mM and 15 mM Vc in FIG. 11A, and 4 mM and 5 mM CA in FIG. 11B. The relative band intensity of DHFR was quantified based on the above western blotting.

### EXAMPLES

In order to make the objectives, technical solutions, and advantages of the present invention clearer, the present invention will be further described in detail below in conjunction with embodiments. The examples are described for the purposes of illustration and are not intended to limit the scope of the invention.

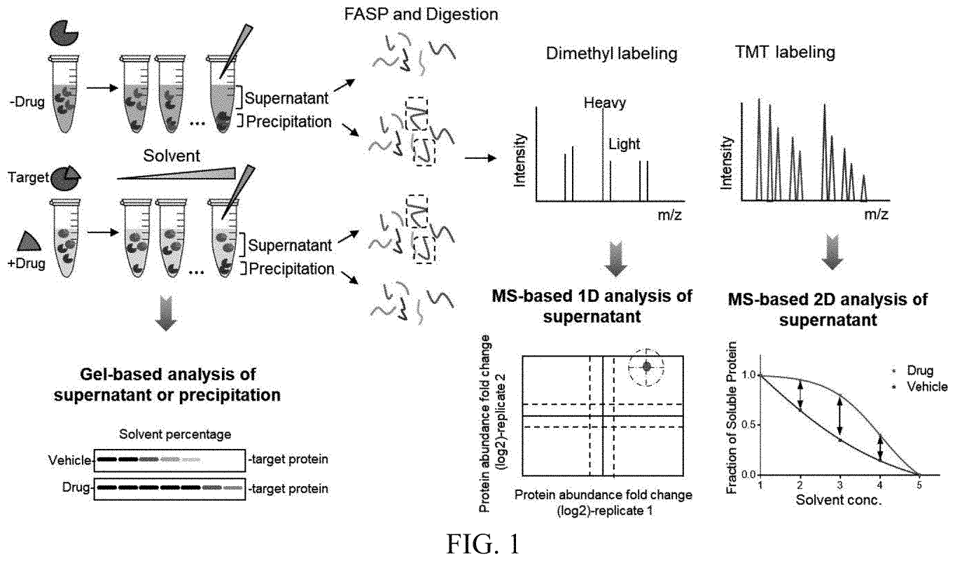

FIG. 1 shows the workflow of solvent-induced protein precipitation method for probing the interaction or affinity between ligands and proteins, which provided by embodiments of the present invention. The workflow is as follows:

Two aliquots of cell lysate are incubated with and without a ligand, respectively. Then the same volume of solvent is added to the two protein solutions to initiate protein precipitation. Solvent mixture A. E. A. (or named A. A. A.) (acetone:ethanol:acetic acid=50:50:0.1, v/v/v) is used as a denaturant to precipitate proteins n the following Examples 1-8. The acidic agent (ascorbic acid (Vc) and citric acid (CA)) are used as denaturants to precipitate proteins in the Examples 9-15. The mixtures are equilibrated at 800 rpm for 20 min at 37° C. with a shaker. After protein precipitation, the soluble proteins are separated from aggregated proteins by centrifugation. The supernatants with/without the ligand are collected for FASP (Filter Aided Sample Preparation) and digestion. Peptide labeling is labeled with stable isotope dimethyl labeling or neutron-encoded multiplex labeling reagent (TMT10). In the dimethyl-labeled samples, the peptides in the supernatant of the ligand group are labeled with heavy labeling, whereas the peptides in the supernatant of the control group are labeled with light labeling. The two differentially labeled digests are mixed and then perform proteomic analysis. Targets were identified through comparing the abundance difference of the same protein between the drug-treated group and the control group at the same concentration of solvent. In some embodiments, all the samples were analyzed by LC-MS/MS replicate. The fold change ratio (log 2 FC) of H/L (heavy standard/light standard) of protein abundance quantified in two replicates is greater than or equal to 1, which is defined as the direct binding protein of the ligand, and the log 2 FC is less than or equal to −1 is defined as a ligand indirect binding protein. In the TMT10 multiple labeling experiment, the resulted 10 labeled peptide extracts in control group and the ligand group are pooled to a single sample per experiment, and then is fractionated into 15 fractions by high-pH reversed phase chromatography for quantitative proteomic analysis. In addition to stable isotope labeling-based method, proteins could also be quantified by label free methods including data independent acquisition (DIA). For the TMT10 labeled samples, targets were identified through comparing the difference in distances sum (ΔDistance) of the same protein in the ligand group and the control group at five denaturation points. Regardless of the above-mentioned labeling or data processing methods, the threshold of the abundance ratio or ΔDistance is not fixed. In theory, the stricter the threshold, the higher the confidence of the target protein identification.

The preparation method of cell lysate is as follows: 10% fetal bovine serum (FBS) and 1% streptomycin were added to RPMI1640 medium at 37° C. and 5% CO2 to incubate HeLa and 293T cells. The harvested cells were washed with cold PBS three times. Subsequently, a final volume concentration of 1% EDTA-free protease inhibitor in PBS (pH 7.4) was added, and then obtain a cell suspension. The cell suspension was frozen with liquid nitrogen, then thawed in a water bath at 37° C. to approximately 60% of the total volume, transferred to ice to continue thawing, and the freeze-thaw process was repeated three times. Cell debris is then removed by centrifuging at 20,000 g for 10 min at 4° C. and the 293T and HeLa cell lysate were obtained.

Specific examples of the method for probing the interaction or affinity between ligands and proteins based on solvent-induced protein precipitation provided in the embodiments of the present invention are as follows:

### Example 1

### Validation of the Solvent Mixture-Based SIP Method by Model Drugs MTX

293T cell lysate was divided into two aliquots of 700 ul, one aliquot was treated with a final concentration of 100 μM MTX as drug group, and the other aliquot was treated with an equivalent amount DMSO alone as the control group, followed by the incubation for 20 min at 10 rpm at room temperature. The cell lysates of the drug-treated group and the control group were divided into 7 EP tubes (100 μL in each EP tube), respectively, and new preparation of different percentages solvent mixtures A.E.A. (the final volume percentage of 9%, 11%, 13%, 15%, 16%, 17% and 18%) was added to the 7 samples to initiate protein precipitation. Subsequently, the mixtures were equilibrated at 800 rpm for 20 min at 37° C. for precipitation. The supernatants were collected after the mixtures were centrifuged at 20,000 g for 10 min at 4° C. One portion was used for western blotting analysis and the left portion was used for MS-based quantification.

The proteins in supernatants were separated by means of SDS-PAGE and were transferred onto a polyvinylidence difluoride (PVDF) membrane. The membrane was blocked with 5% skim milk, and then incubate with DHFR (Subway, China) primary antibody at 4° C. overnight, and secondary goat anti-rabbit HRP-IgG antibody (Abcam, UK) at room temperature for 1 h (The dosage of primary antibody and secondary antibody should be operated according to the manufacturer's instructions). Finally, the chemiluminescence intensities were visualized and quantified by the ECL detection kit (Thermo Fisher Scientific, America) and Fusion FX5 imaging system (Vilber Infinit, France). The other supernatant including the protein samples with or without ligands were processed with filter-aided sample preparation (FASP) technique for proteomics analysis. After addition of a final concentration of 20 mM DTT (Sigma-Aldrich, USA) and 40 mM IAA (Sigma-Aldrich, USA), trypsin (Sigma-Aldrich, USA) was added to the samples (enzyme/protein=1:20, w/w) for digestion at 37° C. for 16 h. The peptides in the control group were labeled with a final volume concentration of 4% CH2O and 0.6 M NaCNBH3 (lightly labeled, L) (Sigma-Aldrich, USA), while the peptides in drug group were labeled with a final volume concentration of 4% CD2O and 0.6 M NaCNBH3 (heavy labeling, H) (Sigma-Aldrich, USA). Subsequently, the two differentially labeled digests in 13% and 15% A.E.A. treated samples were respectively mixed and then subjected to desalt with a C18 solid-phase extraction (Waters, Milford, Mass.). Finally, the peptide samples were resolved in a volume concentration of 1% formic acid (FA) and were analyzed by Ultimate 3000 RSLCnano system coupled with a Q-Exactive-HF mass spectrometry (Thermo Fisher Scientific, America), controlled by Xcalibur software v2.1.0 (Thermo Fisher Scientific, Waltham, Mass., USA). After data processing, the proteins with significant thermal shifts are considered as the candidate targets for drugs.

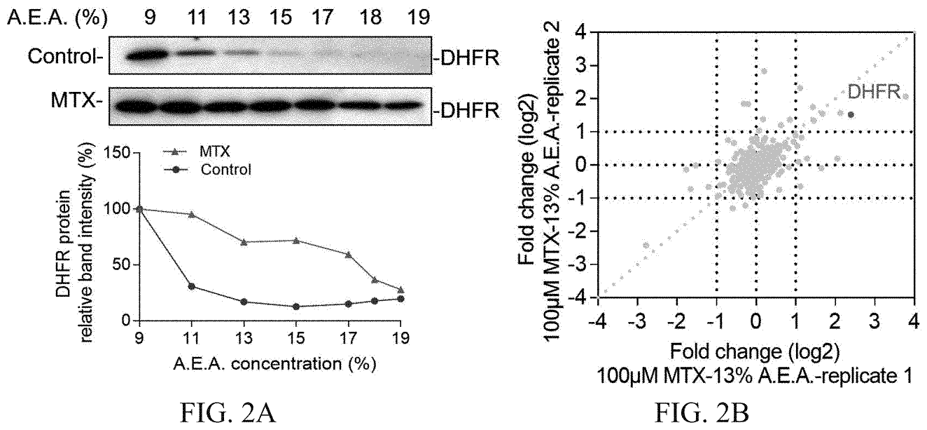

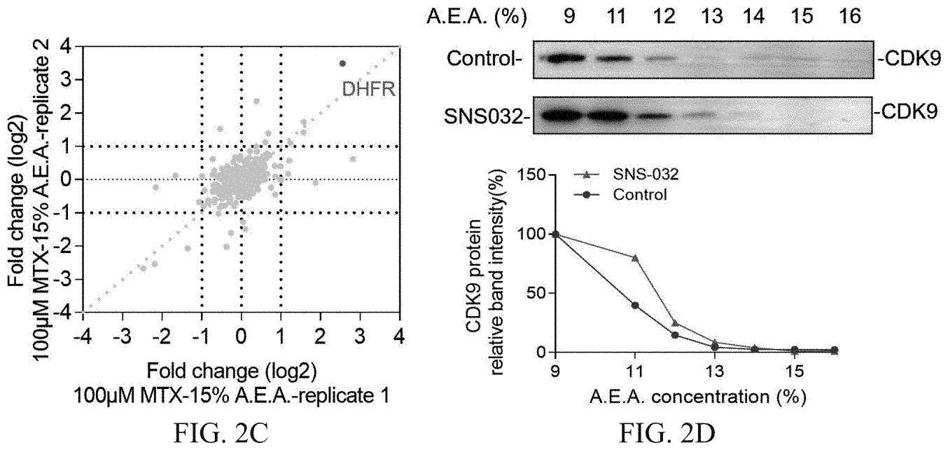

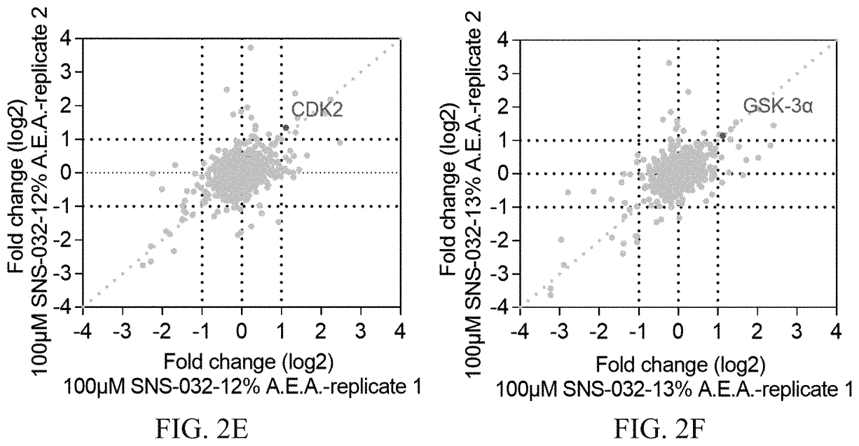

The western blotting result showed the known target protein DHFR bound to the MTX exhibited significant stabilization shifts relative to the ligand-unbound protein under conditions of high percentage A.E.A. (FIG. 2A). Next, the samples treated with 13% and 15% A.E.A. in the control group and the drug-treated group were subjected to quantitative proteomics. The result showed that DHFR was identified in the two labeled samples and the maximum average log 2 FC_{(H/L normalization ratio)} nearly reached 4, indicating significant stabilization after the binding of the drug (FIGS. 2B and C). This proteomics readout was well consistent with the result of western blotting.

The above results showed that the solvent mixture-based SIP method successfully identified the known target protein of the drug MTX, indicating this method can specifically identify the target protein of the drug.

### Example 2

### Validation of the Solvent Mixture-Based SIP Method by Kinase Inhibitor SNS-032

The process and conditions are the same as in Example 1. The difference from Example 1 is that the drug used for verification of known targets is the kinase inhibitor SNS-032 (Selleck, Houston, Tex.). 293T cell lysate was divided into two 700 ul aliquots, one aliquot was treated with the final drug concentrations of 100 μM SNS-032 as drug group and the other aliquot was treated with an equivalent amount DMSO alone as the control group, followed by the incubation for 20 min at 10 rpm at room temperature. The cell lysates of the drug group and the control group were divided into 7 EP tubes (100 μL in each EP tube), respectively, and new preparation of different percentages solvent mixtures A.E.A. (the final volume percentage of 9%, 11%, 12% 13%, 14%, 15%, and 16%) was added to the 7 samples to initiate protein precipitation. Subsequently, the mixtures were equilibrated at 800 rpm for 20 min at 37° C. for precipitation. The supernatants were collected after the mixtures were centrifuged at 20,000 g for 10 min at 4° C. One portion of the supernatants was used for western blotting analysis and the left portion was used for MS-based quantification. The difference of the western blot procedure from Example 1 is that the primary antibody is CDK9 (the amount of antibody used is in accordance with the manufacturer's instructions). The final volume concentration of 12% and 13% A.E.A treated samples were used for mass spectrometry detection. The procedure were the same as those in Example 1.

The western blotting result showed that the CDK9 binding with SNS-032 exhibited significant stabilization shifts relative to free CDK9 when treatment with high percentage of A.E.A. (FIG. 2D). Subsequently, the two samples treated with 12% and 13% A.E.A. in the control group and the drug-treated group were subjected to quantitative proteomics analysis. Two known targets CDK2 and GSK-3a towards drug SNS-032 were identified (FIGS. 2E and 2F), and the average log 2 FC_{(H/L, normalization ratio)} were 1.23 and 1.13, respectively (FIGS. 2E and F), indicating that the protein abundance of known target proteins is greater than 2 fold change.

The above results showed that the solvent mixture-based SIP method successfully identified the known target protein CDK9, CDK2 and GSK-3a of the drug SNS-032, which confirmed that the solvent mixture-based SIP method was able to identify drug targets with high specificity in complex protein samples.

### Example 3

### Identify of Protein Kinase Targets of Pan-Kinase Inhibitor Staurosporine by Solvent Mixture-Based SIP Method

The above drugs only have a few known target proteins. Next, the inhibitor staurosporine, which is known to have multiple protein kinase targets, was selected to verify the feasibility of the solvent mixture-based SIP method. 293T cell lysate was divided into two 300 ul aliquots, one aliquot was treated with final concentration 20 μM staurosporine (Selleck, Houston, Tex.) as drug group and the other aliquot was treated with an equivalent amount DMSO alone as the control group, followed by the incubation for 20 min at 10 rpm at room temperature. The cell lysates of the drug group and the control group were divided into 3 EP tubes (100 μL in each EP tube), respectively, and new preparation of different percentages solvent mixtures A.E.A. (the final volume percentage of 15%, 16%, and 17%) was added to the 3 samples to initiate protein precipitation. Subsequently, the mixtures were equilibrated at 800 rpm for 20 min at 37° C. for protein precipitation. The supernatants were collected after the mixtures were centrifuged at 20,000 g for 10 min at 4° C. and used for MS-based quantification. The procedure of mass spectrometry detection was outlined above in the detail information of Example 1.

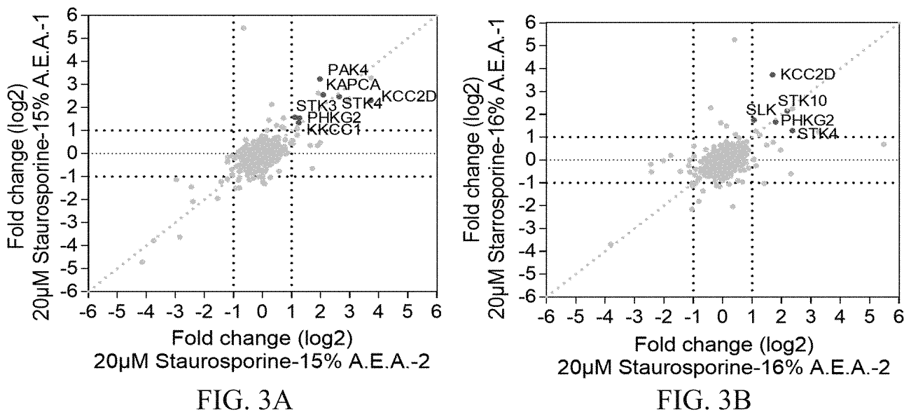

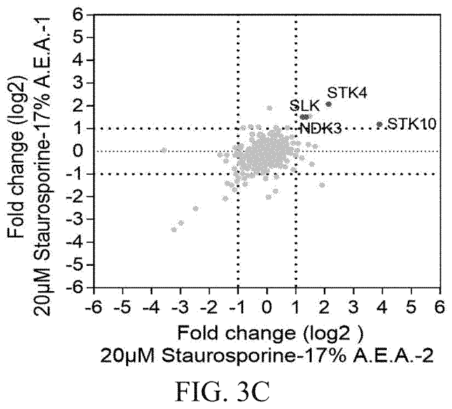

In total, 13, 9 and 5 proteins displayed stabilization shifts were identified in 15%, 16% and 17% A.E.A. samples by proteomic technique. A total of 7, 5 and 4 protein kinases were identified among them and the kinase target hit rates were 58%, 55% and 80%, respectively (FIGS. 3A, B, and C). Although the percentage of kinases in all quantitative proteins only accounted for 5.14%, 4.92%, and 4.98%, the high percentage of protein kinases identified with significant stabilization shifts indicated the high reliability of the solvent mixture-based SIP method. It was found that STK4 kinase was identified in all the three different percentages of A.E.A.-treated samples. Several kinases such as SIK, KCC2D, STK10 and PHKG2 were identified twice among the three samples.

The above results showed that the solvent mixture-based SIP method can screen target proteins of broad-spectrum kinase inhibitor staurosporine in complex samples. The higher kinase target hit rate by identifying the targets of broad-spectrum kinase inhibitor staurosporine further confirmed the high confidence and specificity of the solvent mixture-based SIP method.

### Example 4

### The Consistency and Complementarity of Solvent Mixture-Based SIP and TPP Methods by Using Staurosporine

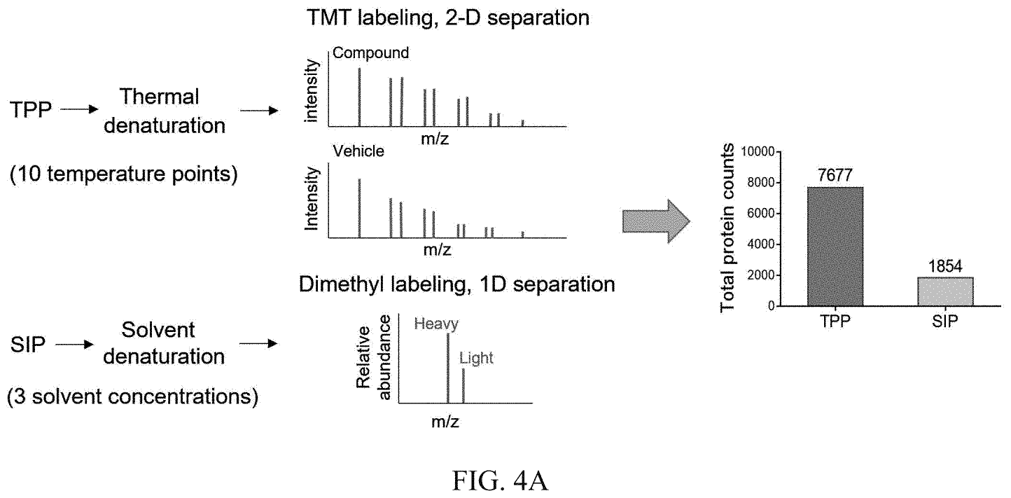

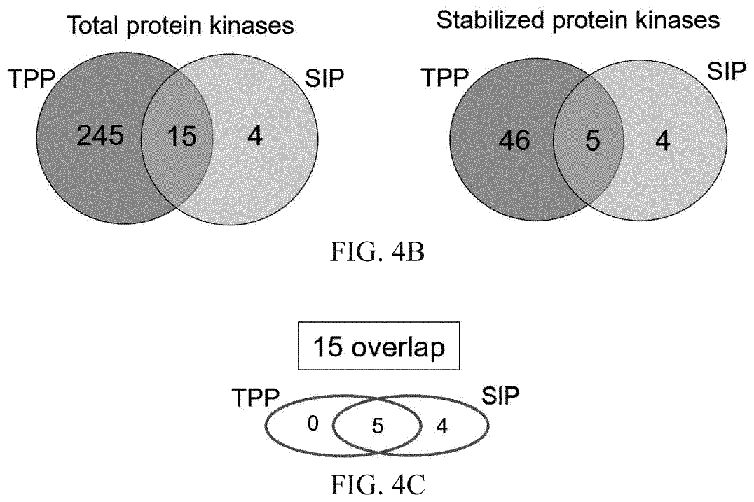

The datasets of this embodiment were acquired from Example 3. Savitski et al, ((2014) Science 346: 1255784) have performed a comprehensive study to screen the targets of staurosporine by TPP approach. In their study, the samples from 10 different temperatures were labeled with the neutron-encoded isobaric mass tagging reagents (TMT10) and analyzed with 2D RP-RPLC MS/MS, which resulted in the quantification of 7677 proteins (FIG. 4A). In total, the thermal profiles comprised 260 protein kinases, of which 51 (19.62%) were determined to be the target proteins according to the melting curves (FIG. 4B). While in this study, the samples from three different solvent concentrations were separately subjected to dimethyl labeling and 1D RPLC MS/MS analysis, which quantified only 1854 proteins (FIG. 4A). Due to the poor proteome coverage, only 19 protein kinases were quantified in this study. Among them, 47.37% (9/19) were found to be significantly stabilized after binding of drug and determined to be the potential target proteins (FIG. 4B). Because the high proteome coverage of the TPP studies, 15 protein kinases quantified in this study were included in the 260 protein kinases quantified in Savitski-TPP (FIG. 4B, left). As these 15 protein kinases were quantified in both studies, it is of interest to see if they showed stabilization shift in both cases. It was found that only 5 protein kinases had significant stabilization shifts in both Savitski-TPP and solvent mixture-based SIP (FIG. 4C). There were 4 protein kinases including SIK, KCC2D, STK10 and KKCC1 showed significant stabilization only in solvent mixture-based SIP approach but not in Savitski-TPP approach and no protein was found to have significant stabilization shift only in Savitski-TPP approach (FIG. 4C). Therefore, above data indicated different precipitation methods, in addition to the common target proteins, some candidate proteins are complementary.

The above results indicated that the solvent mixture-based SIP and the Savitski-TPP methods were consistent and complementary in target protein identification.

### Example 5

### Discovery of Protein Targets of Geldanamycin by Solvent Mixture-SIP Method and Validation of Potential Off-Target Protein

Hela cell lysate was divided into two 700 ul aliquots, one aliquot was treated with 100 μM geldanamycin (Selleck, Houston, Tex.) as drug group and the other aliquot was treated with an equivalent amount DMSO alone as the control group, followed by the incubation for 20 min at 10 rpm at room temperature. The cell lysates of the a drug group and the control group were divided into 7 EP tubes (100 μL in each EP tube), respectively, and new preparation of different percentages solvent mixtures A.E.A. (the final volume percentage of 9%, 12%, 13%, 14%, 15%, 16% and 17%) was added to seven samples to initiate protein precipitation. Subsequently, the mixtures were equilibrated at 800 rpm for 20 min at 37° C. for protein precipitation. The supernatants were collected after the mixtures were centrifuged at 20,000 g for 10 min at 4° C. The supernatant was used for MS-based quantification. The procedure used for western blotting detection differs from Example 1 is that the antibodies are HSP90AB1 and NDUFV1 (The amount of antibody used was in accordance with the manufacturer's instructions). Samples treated with 15%, 16% and 17% A.E.A. were subjected to dimethyl labeling for MS analysis, the procedure were the same as those in Example 1.

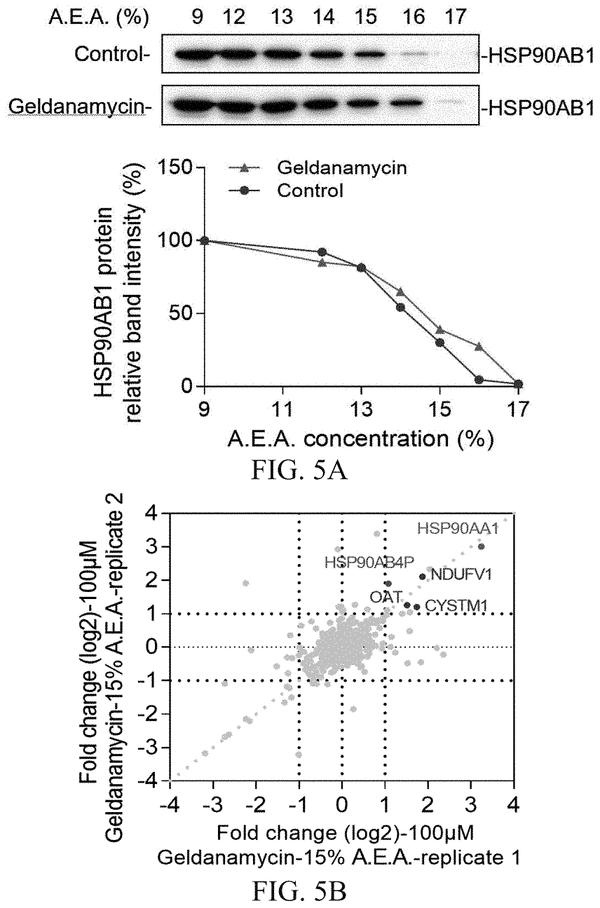

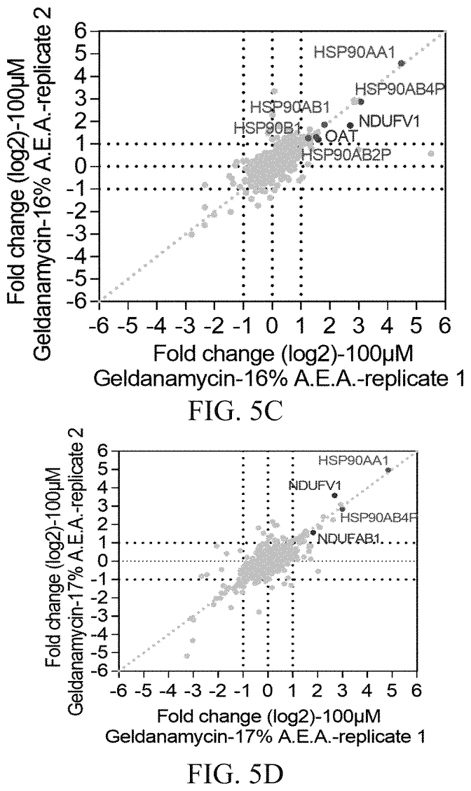

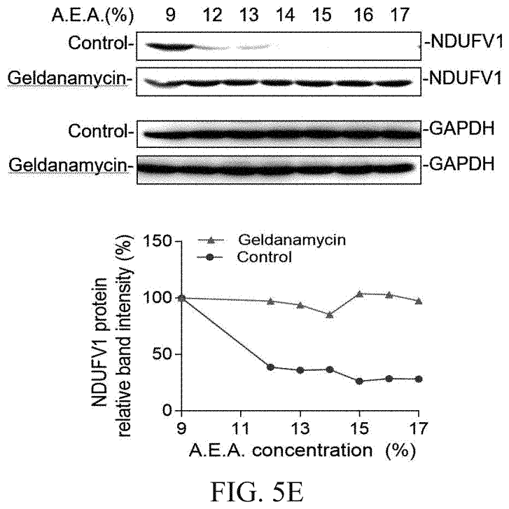

It can be seen that HSP90AB1 started to precipitate when A.E.A. percentage increased from 15% to 17% (FIG. 5A). And it was obvious that the HSP90AB1 binding with drug was more resistant to A.E.A. induced precipitation (FIG. 5A). The three known protein targets in HSP90 family of geldanamycin, were successfully identified in 15%, 16% and 17% A.E.A.-treated samples by quantitative proteomics analysis (FIGS. 5B, 5C and 5D). HSP90AA1 was reproducibly identified among these three samples with different A.E.A. percentages, and the three known protein targets were mainly concentrated in the samples with 16% A.E.A. (FIG. 5C). The other HSP90 isoforms such as HSP90AB2P and HSP90AB4P were also reproducibly identified. Furthermore, several proteins were first identified as off targets, including the subunits of the mitochondrial membrane respiratory chain NADH dehydrogenase such as NDUFV1 and NDUFAB1 (FIG. 5D). The western blotting illustrated that the abundance of free NDUFV1 protein decreased significantly at 12% percentage of A.E.A, while its abundance with geldanamycin kept constant even with the highest percentage of A.E.A. (17%) (FIG. 5E).

The above results indicated NDUFV1 is a high confident off-target of geldanamycin. Therefore, solvent mixture-SIP method is capable of screening high-confidence unknown targets of a ligand.

### Example 6

### Evaluating Affinity for Geldanmycin-HSP90AB1 Interaction Using Solvent Mixture-based SIP Method

In the affinity detection of drug-target protein interaction, the HeLa cell lysates were incubated with different concentrations of geldanamycin solutions (10¹, 10⁰, 10⁻¹, 10⁻², 10⁻³, 10⁻⁴, 10⁻⁵, 10⁻⁶, 10⁻⁷, 10⁻⁸ and 10⁻⁹ μM) at room temperature for 20 min with 10 rpm rotation. Each sample was treated with final volume concentration 15% A.E.A., followed by the mixtures were equilibrated at 800 rpm for 20 min at 37° C. for protein precipitation. Subsequently, the supernatants were collected after the mixtures were centrifuged at 20,000 g for 10 min at 4° C., and then analyzed by western blotting. The procedure differs from Example 1 is that the antibody was HSP90AB1 (Proteintech, Chicago, Ill.) (The amount of antibody used was in accordance with the manufacturer's instructions).

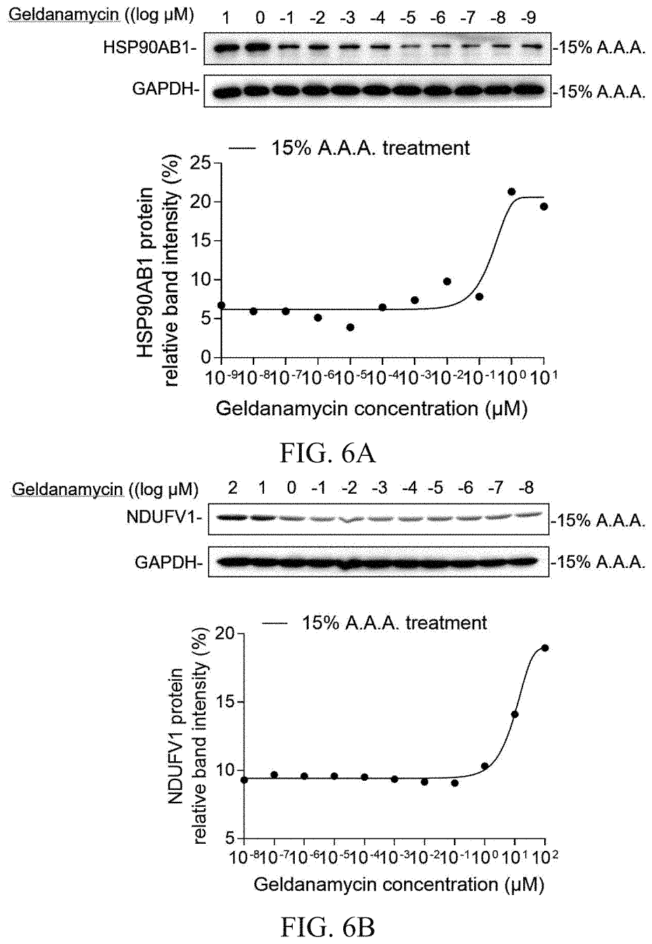

The result showed the abundance of HSP90AB1 obviously decreased from the concentration at 1 μM (FIG. 6A). The half-saturation point of geldanamycin-binding HSP90AB1 complex was between 1 μM and 10 μM concentration, and geldanamycin reached the full occupancy of HSP90AB1 protein around the concentration of 10 μM (FIG. 6A).

Above result showed that this method determine the affinity (binding strength) of geldanamycin and HSP90AB1. Clearly, the solvent mixture-based SIP approach is also able to determine the affinity of drug-protein interaction.

### Example 7

### Evaluating Affinity for Geldanmycin-NDUFV1 Interaction Using Solvent Mixture-Based SIP Method

The procedure and conditions was as same as Example 6, the differences from Example 6 was that, the concentrations of geldanamycin were 10², 10¹, 10⁰, 10⁻¹, 10⁻², 10⁻³, 10⁻⁴, 10⁻⁵, 10⁻⁶, 10⁻⁷, 10⁻⁸ μM; the antibody was NDUFV1 (Proteintech, Chicago, Ill.) (The amount of antibody used was in accordance with the manufacturer's instructions).

Western blotting-based curve confirmed that the half-saturation point of the latent target protein NDUFV1 of geldanamycin was between 10-100 μM. Geldanamycin reached the full occupancy at 100 μM, which was about 10 times higher than that in the interaction between geldanamycin and its known HSP90AB1 proteins (FIG. 6B).

The above results determined the affinity between geldanamycin and NDUFV1. Therefore, the above results indicated that the solvent mixture-based SIP method could determine the affinity of a drug and novel proteins.

### Example 8

### GO and Pathways Analysis for Off-Target Protein of Geldanamycin

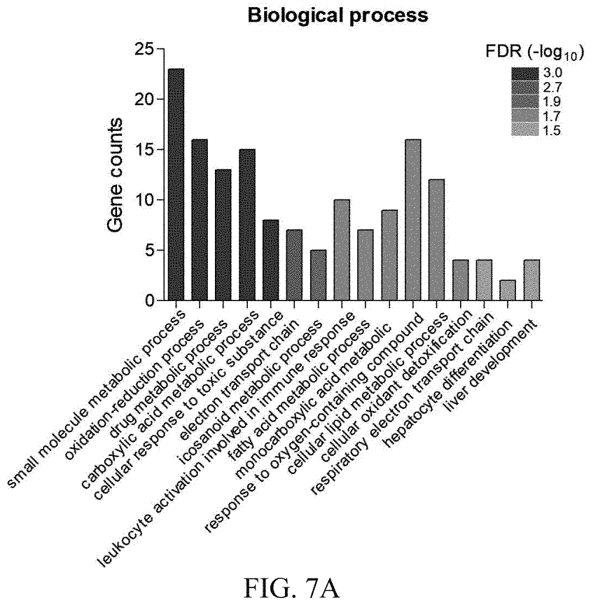

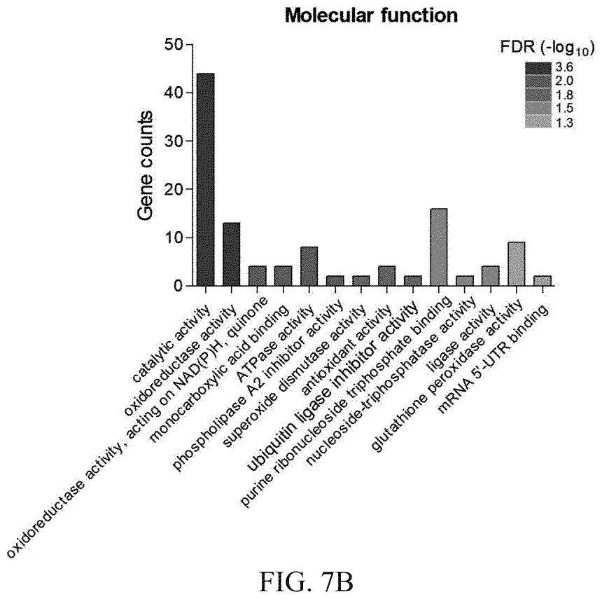

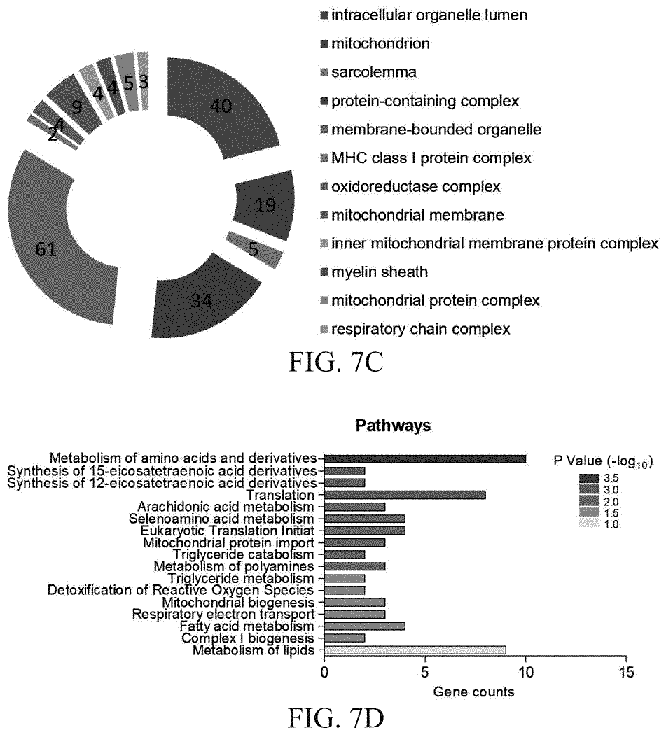

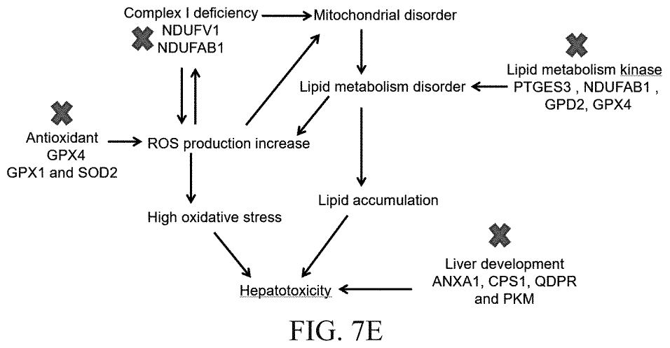

The datasets of this embodiment were acquired from Example 5. Geldanamycin is the pioneering and potent inhibitor of HSP90. However, it was withdrawn from clinical trials due to the serious side effects which lead to severe hepatotoxicity. All stabilized and destabilized proteins hits excluding the known HSP90 family proteins were subjected to Gene ontology and pathways analysis. It was found that most of the protein hits involved in metabolism, oxidation-reduction process and mitochondria function (FIGS. 7A, 7B, 7C and 7D). The above data collectively demonstrated that the hepatotoxicity induced by geldanamycin may be due to the promiscuous off-target effects, involving in mitochondrial respiratory chain disturbances, redox processes, ROS accumulation, metabolic disturbances, and impairment of liver development (FIG. 7E).

The construction of the target space by solvent mixture-based SIP method can reveal the expected targets and off-targets induced side effect.

### Example 9

### Validation of the Vc-Based SIP Method by Model Drugs MTX

293T cell lysate was divided into two 700 ul aliquots, one aliquot was treated with final drug concentrations of 100 μM MTX (Selleck, Houston, Tex.) as drug group and the other aliquot was treated with an equivalent amount DMSO alone as the control group, followed by the incubation for 20 min at 10 rpm at room temperature. The denaturation was initiated by addition of different concentrations of Vc (1 mM, 2 mM, 4 mM, 6 mM, 8 mM, 10 mM and 12 mM. Subsequently, the mixtures were equilibrated at 800 rpm for 20 min at 37° C. for protein precipitation. The supernatants were collected after the mixtures were centrifuged at 20,000 g for 10 min at 4° C. One portion of the supernatants was used for western blotting analysis and the left portion was used for MS-based quantification. The procedure of western blotting was as same as Sample 1. The samples treated with 6 mM and 8 mM Vc were selected for MS analysis, and the procedure was as same as Sample 1.

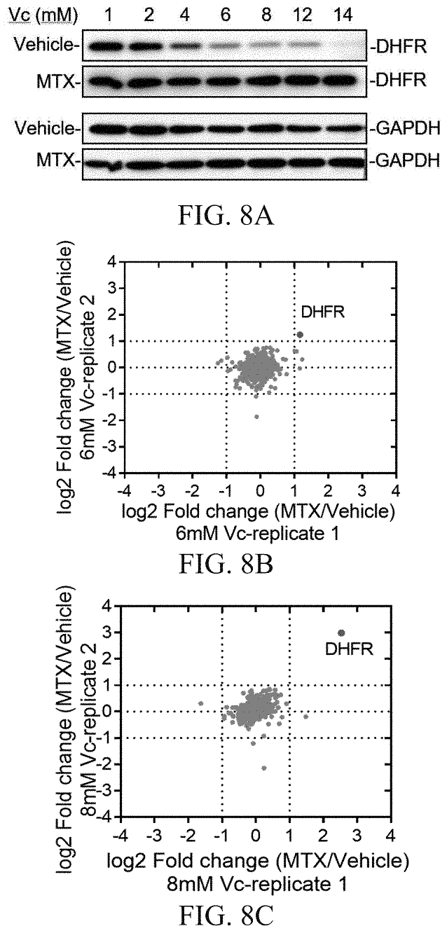

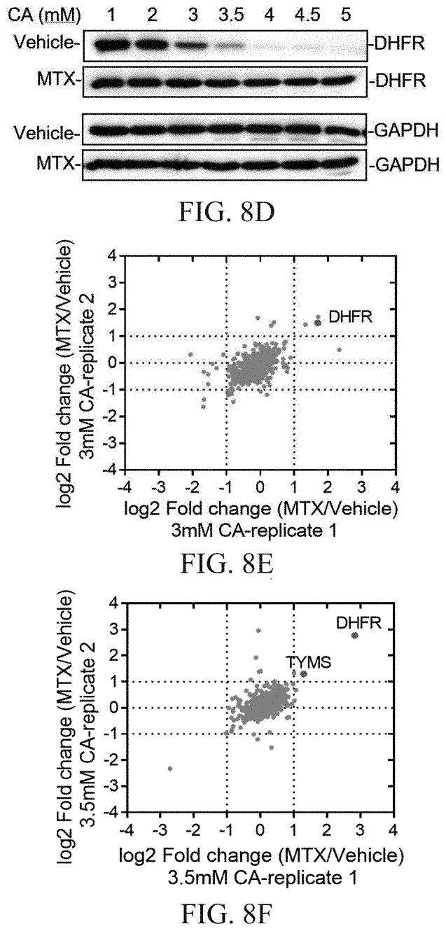

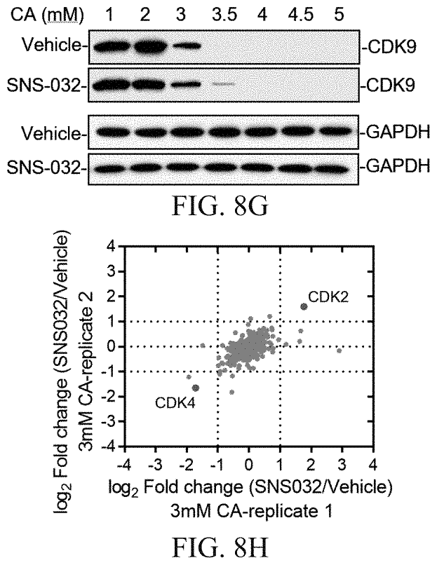

Western blotting result showed that the abundant of target protein DHFR binding with MTX was higher than that in the control group when treatment with Vc concentration above 6 mM, confirming that the DHFR bound to MTX has higher stability shift (FIG. 8A). Next, the samples treated with 6 mM and 8 mM in the control group and the drug-treated group were subjected to digestion and dimethyl labeling for LC-MS/MS analysis. The proteomics readout showed that DHFR was identified as the only protein target in the two labeled samples (FIGS. 8B and 8C). As the Vc concentration increased, the fold change in the expression level of the target protein gradually increased, and the average log 2 FC was 1.21 and 2.76, respectively (FIGS. 8B and 8C), indicating that drug binding induced protein stabilization.

The above results showed that the Vc-based SIP method successfully identified the known target protein of the drug MTX, indicating this method can specifically identify the target protein of a drug.

### Example 10

### Validation of the CA-Based SIP Method by Model Drugs MTX and SNS-032

293T cell lysate was divided into two 700 ul aliquots, one aliquot was treated with final drug concentrations of 100 μM MTX or SNS-032 (Selleck, Houston, Tex.) as drug group and the other aliquot was treated with an equivalent amount DMSO alone as the control group, followed by the incubation for 20 min at 10 rpm at room temperature. The denaturation was initiated by addition of different concentrations of Vc (1 mM, 2 mM, 3 mM, 3.5 mM, 4 mM, 4.5 mM and 5 mM). Subsequently, the mixtures were equilibrated at 800 rpm for 20 min at 37° C. for protein precipitation. The supernatants were collected after the mixtures were centrifuged at 20,000 g for 10 min at 4° C. One portion of the supernatants was used for western blotting analysis and the left portion was used for MS-based quantification. The procedure of western blotting was as same as Sample 1. The samples treated with 3 mM and 3.5 mM CA were selected for MS analysis, and the procedure was as same as Sample 1.

Western blotting result showed that the level of DHFR bound with MTX was higher than that in the control group with the increasing CA concentrations. Especially after the CA concentration above 4 mM, the DHFR protein expression in the control group was almost undetectable, but DHFR still maintained a high level in the drug group (FIG. 8D). Next, the samples treated with 3 mM and 3.5 mM in the control group and the drug-treated group were subjected to quantitative proteomics. The readout showed that DHFR was identified as the top protein target in the two labeled samples. With the CA concentration increased, the fold change in the expression level of the target protein gradually increased, and the average log 2 FC (H/L normalized ratio) was 1.60 and 2.80, respectively (FIGS. 8E and 8F). Interestingly, another target TYMS of MTX was also identified in the 3.5 mM CA-treated sample (FIG. 8F). TYMS is a metabolite in cells, which means that it has a certain activity and metabolic effect after the cells are crudely extracted in vitro. In the SNS-032 experiment, the abundance of the known target CDK9 binding with SNS-032 was higher as compared with that in the control group (FIG. 8G). The 3.5 mM CA-treated sample was performed dimethyl labeling for quantitative proteomics. Although CDK9 protein was not quantified, another target CDK2 was identified as the top target, and the CDK4 was identified as destabilized binding protein (FIG. 8H).

The above results showed that the CA-based SIP method enabled successfully identify the known target DHFR and TYMS of MTX, indicating that this method can specifically identify the target protein of a drug.

### Example 11

### Identify of Protein Kinase Targets of Pan-Protein Kinase Inhibitor Staurosporine by CA-Based SIP Method

Next, a pan-protein kinase inhibitor staurosporine was selected and combined with TMT isobaric labeling-based proteomics methods to further evaluate the CA-based SIP method for target identification. 293T cell lysate was divided into two 700 ul aliquots, one aliquot was treated with final concentration 20 μM staurosporine (Selleck, Houston, Tex.) as drug group and the other aliquot was treated with an equivalent amount DMSO alone as the control group. And followed by the incubation for 20 min at room temperature using rotometer. The denaturation was initiated by addition of different concentrations of CA (2.5 mM, 3 mM, 3.5 mM, 4 mM and 5 mM). Subsequently, the mixtures were equilibrated at 800 rpm for 20 min at 37° C. for protein precipitation. The supernatants were collected after the mixtures were centrifuged at 20,000 g for 10 min at 4° C. and used for trypsin digestion. The resulting peptides of samples treated with 5 concentrations in the control and drug groups were performed TMT10 labeling and the labeling procedure was according to the manufactory's instrument. A total of 10 samples in the control and drug groups were labeled according to the labeling peptide:reagent=1:4 (w/w), and the reaction was shaken at 25° C. and 1000 rpm for 1 h. The reaction was then terminated with 5% hydroxylamine (Sigma-Aldrich, USA), and shaken at 1000 rpm for 20 min at 25° C. The 10-plex labeled samples were pooled together and then fractionated into 15 fractions by high-pH reversed phase chromatography before quantitative proteomics analysis.

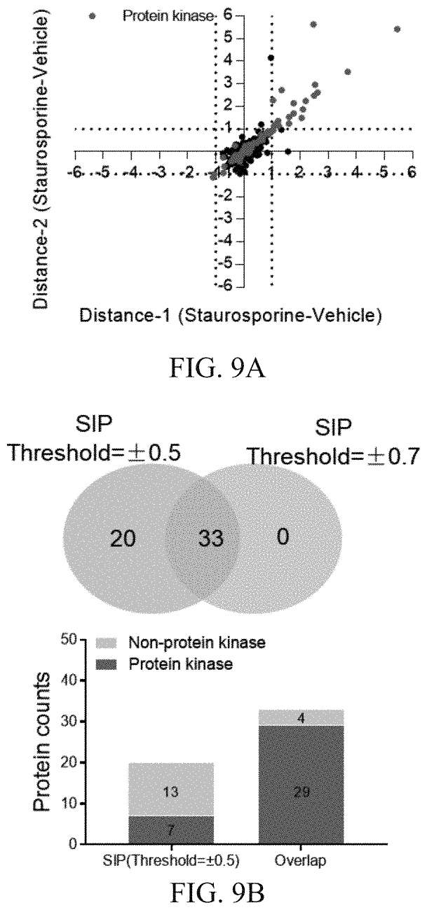

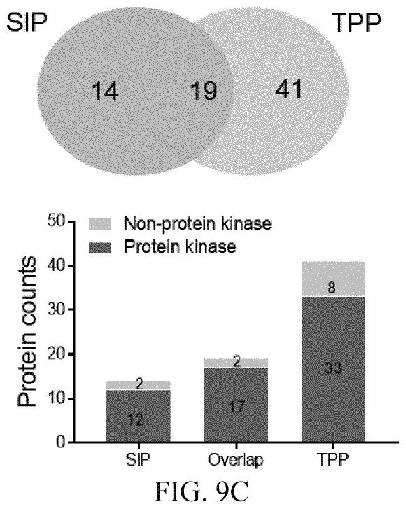

The reporter ion intensities acquired from the control group and the drug group across each denaturation points were normalized by the ratio of median value of proteins in the control group and the drug group. The reporter ion intensities of the 10 datasets were further normalized by the reporter ion intensities of 2.5 mM CA in the control group. The distance at each denaturation point was obtained by the normalized reporter ion intensities of the 5 denaturation points in the drug group minus that in the control group. Ultimately, the sum of distance at each denaturation point (ΔDistance) was reflected the stability shift of a protein upon binding with a ligand. The threshold for screening the sum of ligand-target protein distances is adjusted for different ligands. It can be seen in FIG. 9A that the proteins with significant stability shift were almost protein kinases.

The above results indicate that the CA-based SIP method has a high protein kinase hit rate for the targets identification of staurosporine. Therefore, the above results indicated that the CA-based SIP method has high specificity in target protein identification.

### Example 12

### The Effect of Different Thresholds on Staurosporine Candidate Target Identification

The datasets of this embodiment were acquired from Example 11. In this example, the filtering criterion is defined as a protein with ΔDistance≥0.5 was stabilized proteins and a protein with ΔDistance≤0.5 was destabilized proteins. Both stabilized and destabilized proteins were used as the total candidate target proteins of staurosporine. After filtering, a total of 53 candidate proteins were identified, of which 36 were protein kinases and 17 were non-protein kinases (FIG. 9B). Non-protein kinases accounted for 32% of the total candidate binding proteins.

In this embodiment, a protein with the threshold of ΔDistance≥0.5 was stabilized proteins and a protein with ΔDistance≤0.5 was destabilized proteins. Both stabilized and destabilized proteins were used as the total candidate target proteins of staurosporine. After filtering, a total of 33 candidate binding proteins were identified, of which 29 were protein kinases and 4 were non-protein kinases (FIG. 9B). Non-protein kinases accounted for 12% of the total candidate binding proteins.

The above results indicated that the more rigorous the threshold, the higher confidence of the candidate target proteins.

### Example 13

### The Consistency and Complementarity Between CA-Based SIP and TPP Methods by Using Staurosporine

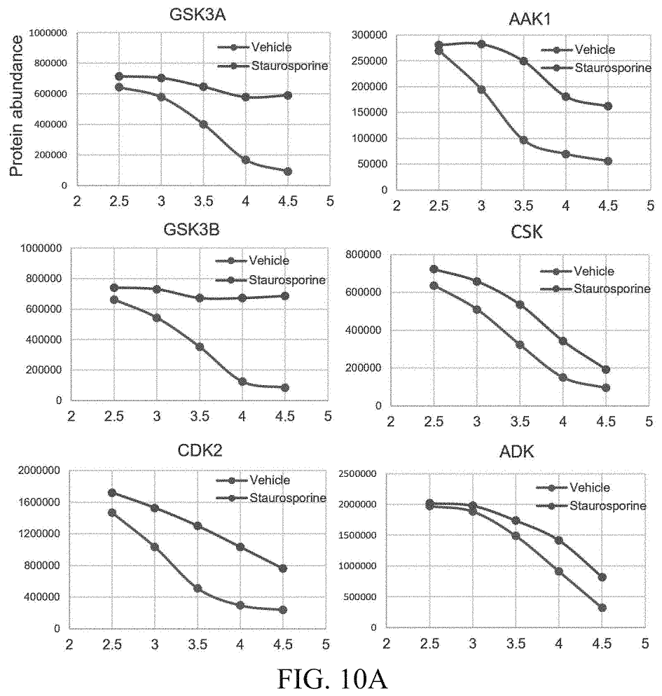

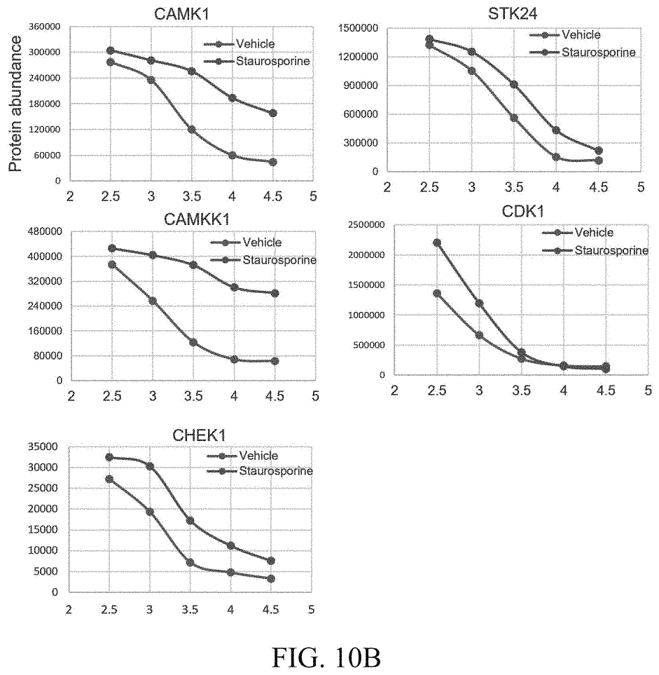

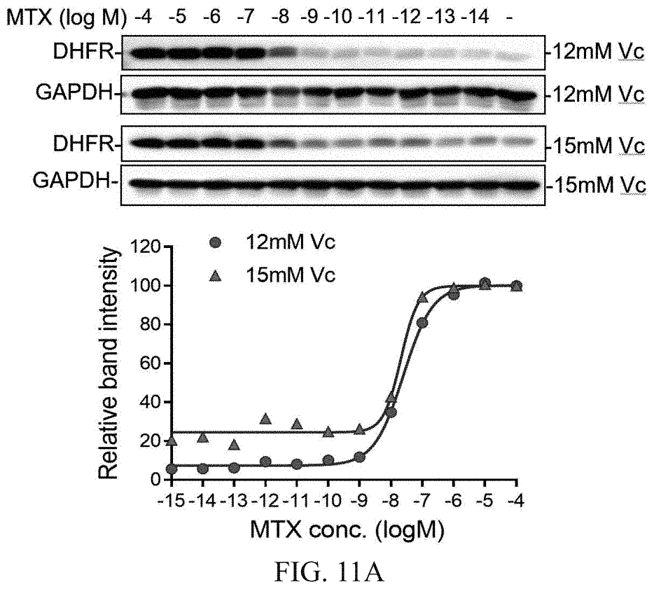

The datasets of this embodiment were acquired from Example 11. In order to understand the differences between CA-based SIP and Savitski-TPP methods in target proteins identification, the protein kinase targets identified by CA-based SIP method was compared with Savitski-TPP method. In this example, the filtering criterion is defined as a protein with ΔDistance≥0.7 was stabilized protein, instead, a protein with ΔDistance≤0.7 was destabilized protein. Both stabilized and destabilized target protein were used as the total candidate protein targets of staurosporine. After filtering, 33 candidate proteins were obtained, whereas the Savitski-TPP method identified 60 candidate proteins. The reason was the difference of protein coverage by Savitski-TPP and CA-based SIP methods. In the CA-based SIP method, a total of 3636 proteins were identified from 5 concentration points in two mass spectrometry replicates, of which 103 were protein kinases, while the TPP method identified a total of 7677 proteins, of which 260 were protein kinases. A total 19 staurosporine-induced stabilized proteins were commonly identified in CA-based SIP and Savitski-TPP methods, of which 17 were protein kinases such as GSK3-β, CDK2 and AAK1 (FIG. 9C and FIG. 10A). Among them, 14 proteins were only identified in the SIP method but not in the Savitski-TPP method, including 12 protein kinases such as CAMK1, CDK1 and CHECK1 (FIG. 9C and FIG. 10B).

The above results indicated that the target proteins of a ligand identified by the CA-based SIP method were complementary with TPP method.

### Example 14

### Evaluating Affinity for MTX and the Known Target DHFR Using Vc-Based SIP Method

In order to evaluate whether the acidic agent-based SIP method can be applied to the determination of affinity of drug with the target protein, MTX was used to perform drug dose-dependent response experiment by using Vc as the denaturant. The 293T cell lysate was incubated with different concentrations of MTX at room temperature at 10 rpm for 20 minutes, and then the protein was denatured and precipitated with 12 mM and 15 mM Vc, respectively. After the reaction was equilibrated at 37° C. and 800 rpm for 20 minutes for protein precipitation, the cell lysate was centrifuged to separate the supernatant from the precipitate at 4° C., 20,000 g for 10 min. The supernatant was collected for western blotting detection, and the procedure was as same as Sample 1.

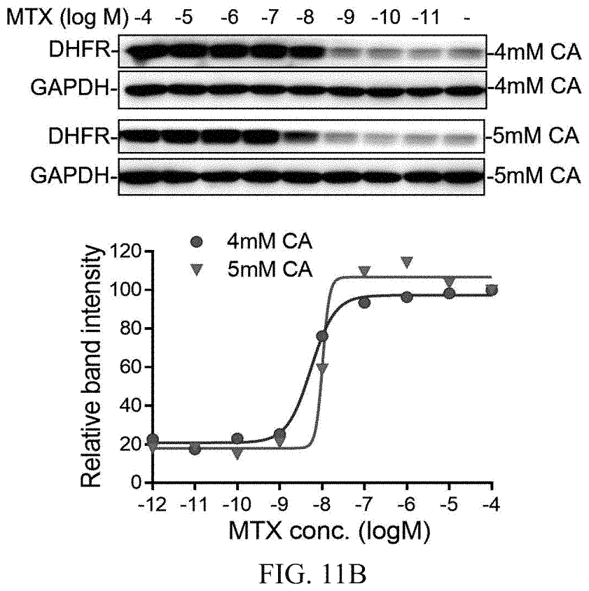

The western blotting result showed that the relative band intensity of target DHFR showed an upward trend with the increase of MTX dose, and the intensity of DHFR obviously decreased from 10 uM (10⁻⁸ M) (FIG. 11A). The dose-dependent response curve showed that the half-saturation midpoint of DHFR to MTX was around 10 uM, and MTX fully occupied the DHFR protein and reached a saturated state (maximum stability state) at a concentration of around 100 μM, This result was consistent with the known EC50 of MTX to DHFR (FIG. 11A).

The above result showed that the Vc-based SIP method enables determine the affinity of MTX and the known target DHFR, indicating that this method can determine the affinity between the ligand and the target protein.

### Example 15

### Evaluating Affinity for MTX and the Known Target DHFR Using CA-Based SIP Method

The difference from Example 14 was that 4 mM and 5 mM CA were used as the denaturant to determine the affinity of MTX with the known target protein DHFR. The western blotting readout showed that the half-saturation midpoint of DHFR to MTX was around 10 μM (FIG. 11B), which was similar to the known EC50 of MTX to DHFR. DHFR saturated when MTX concentrations reach around 100 μM, which approximated the known EC50 of MTX.

The above results indicated that the CA-based SIP method can evaluate the affinity between MTX and DHFR. Therefore, the above results in Example 14 and 15 indicated that the acidic agent-based SIP method could determine the affinity between the ligand and the interacting target protein.

What is claimed:

1. A method for detecting the interaction between a ligand and a protein based on solvent-induced protein precipitation, wherein this method is established by exploiting the tolerance difference of protein with and without a ligand to solvent-induced protein precipitation for target identification. After the equal amount of solvent is added to the protein samples with and without a ligand to denature and precipitate the proteins, the protein abundances in supernatant and/or precipitate in the ligand group and the control group are measured by quantitative technology. The target protein(s) of a ligand is/are determined by comparing the differences of protein abundances in the ligand group and the control group.

2. The method of claim 1, wherein the method comprising:
(a) The protein solution incubated with a ligand is used as ligand group, the protein solution incubated with equivalent amount of ligand dissolving solvent in the absence of ligand is used as the control group;
(b) Add an equal amount of denaturing solvent to the ligand group and the control group to initiate protein denaturation and resulting in precipitation.
(c) Quantify the abundance of each protein in supernatant and/or precipitate of the ligand group and the control group.
(d) Compare the abundance difference of each protein in the ligand group and the control group (that is, the difference in the abundance of the same protein in the supernatant and/or precipitate) to determine the target(s) of a ligand.

3. The method for determining affinity between a ligand and target protein based on solvent-induced protein precipitation.
(a) Incubate the same ligand with different concentration gradients with the tested protein solution separately (usually choose 5 or more final concentrations of the ligand, one of which is the control point without ligand, that is, the final concentration of the ligand at the control point is 0);
(b) Add an equal amount of solvent (preferably the same final concentration) to the mixture containing proteins and ligand to precipitate the proteins;
(c) Quantify the abundance of each protein in supernatant and/or precipitate containing proteins and ligand.
(d) Calculate the concentration for 50% of maximal effect (i.e. EC50) to obtain affinity between the ligand and the target protein, by taking the abscissa as the different drug concentrations and the ordinate as the protein abundance to fit curve. The calculation equation: Y=min+(max−min)/(1+10\hat{ }((Log EC50−X)*Hill Slope)), Y is the protein abundance, X is the different drug concentrations, min and max are the minimum and maximum values of the corresponding protein abundance on the Y axis, respectively. Hill Slope is the absolute value of the maximum slope of the curve (i.e., the midpoint of the curve).

4. The method of claim 2, wherein the soluble protein (supernatant) is separated from the precipitate by centrifugation before the quantification in step (c).

5. The method of claim 2, wherein the protein solution includes one protein or a mixture of two or more proteins; the protein mixture includes one or more of cell or tissue extracts; the cell or tissue extract is derived from one or more of humans, animals, plants or bacteria.

6. The method of claim 2, wherein the protein solution includes one or more of blood or plasma; the blood or plasma is derived from one or more of humans or animals.

7. The method of claim 2, wherein the protein solution adopts mild extraction condition to allow proteins in one or more cells or tissues to maintain the natural conformation (the specific spatial structure of the protein in living cells or tissues);
Preferably, the extraction condition includes, but not limit to, PBS (phosphate buffer saline) alone or PBS supplemented with 0.2-0.4% NP-40 (Nonidet P 40) as a buffer, combining with three freeze-thaw cycles in liquid nitrogen. The thawing temperature is at 10-50° C.

8. The method of claim 2, wherein the ligand includes one or more of drugs, metabolites, natural productions or plant extracts, food additives, environmental pollutants, agriculture pesticides or herbicides, environmental agents, metal ions, nanoparticles, peptides, proteins and other substances that may interact with proteins.

9. The method of claim 1, wherein the protein solution is divided into two groups in step (a), one group is added with a ligand as the ligand group, and the other group is without ligand as the control group;
Or, but not limited to two groups. The ligand group can perform more than two groups of protein samples with different concentrations of ligand, and the control group can apply blank (that is, without ligand(s)) or other ligand(s) with similar structure(s) and different target proteins.

10. The method of claim 1, wherein the solvent for protein denaturation is one or a mixture of two or more solvents;
wherein the solvent include one or more of organic or inorganic substances that can denature and precipitate proteins. The solvent includes but not limited to one or more of solvents, acidic agents, alkaline agents, metal ions or salts.

11. The method of claim 10, wherein the solvent includes, but not limited to, one or two of acetone, methanol, ethanol, acetic acid, ascorbic acid (Vc), citric acid (CA), and trifluoroacetic acid.
Preferably, the solvent mixture includes, but is not limited to, a mixture of three solvents (acetone, ethanol and acetic acid) (abbreviated as A. E. A. or A. A. A.). The volume ratio of solvent mixture is acetone:ethanol:acetic acid=50:50:0.1.

12. The method of claim 1, wherein the solvent window used for protein denaturation and precipitation in the ligand group and the control group can be appropriately adjusted according to the different solvents. The principle of solvent window selection is that the solvent or solvent mixture can cause the initial precipitation of the protein to the range of 80-90% of the protein precipitation.
Preferably, the final volume percentage of the solvent mixture A.E.A. (or A.A.A.) within the range of 9%-22%; the final concentration of Vc is within the range of 1-15 mM; or, the final concentration of CA is within the range of 1-5 mM.

13. The method of claim 2, wherein the equilibrium condition of the solvent-treated protein solution can be adjusted appropriately (that is, shaking at 20-30° C. for 20-40 min or shaking at 30-40° C. for 10-20 min to achieve the purpose of partial protein denaturation in protein solution).
Preferably, the condition of reaction equilibrium is shaking at 800 rpm for 20 min at 37° C.

14. The method of claim 2, wherein the protein abundance can be detected in the soluble fraction (i.e., the supernatant) or precipitate, or in both.
wherein the methods to quantify the protein abundance in the ligand group and the control group after the solvent(s) treatment in step (c) include, but are not limited to western blotting and quantitative proteomics technology;
wherein the labeling methods of peptides in quantitative proteomics technology include label-free quantification and/or label quantification;
wherein the methods of labeling quantification include one or more of dimethyl labeling and multiplex isotope labeling methods such as TMT (neutron-encoded isobaric tandem mass tags) or ITRAQ (isobaric tags for relative and absolute quantification);

15. The method of claim 2, wherein the methods to quantify the protein abundance in the ligand group and the control group after the solvent(s) treatment include, but are not limited to one or more 1D electrophoresis, 2D electrophoresis, western blotting and mass spectrometry;
wherein the mass spectrometry-based quantification methods include, but are not limited to Data Dependent Acquisition (DDA), Data Independent Acquisition (DIA), Selected reaction monitoring (SRM) and Multiple reaction monitoring (MRM).

16. The method of claim 2, wherein the methods to compare the stabilization shift of the same protein both in the ligand group and the control group include calculating their difference in the abundance or relative abundance distance.

17. The method of claim 2, wherein the criterion for target protein identification is that the difference in the abundance or relative abundance distance of each protein in the ligand group and the control group is ≥ or ≤ to a certain threshold.
The threshold can be adjusted appropriately according to the different ligands, peptide labeling methods or quantitative proteomics technologies (determine the optimal threshold by maximizing the sensitivity and specificity, such as the fold change of protein abundance ≥2).
